(19) United States
(12) Patent Application Publication
(10) Pub. No.: US 2018/0228885 A1
(43) Pub. Date: Aug. 16, 2018

(54) BACTERIAL AND VIRAL VACCINE STRATEGY

(71) Applicant: ORBIS HEALTH SOLUTIONS LLC, Greenville, SC (US)

(72) Inventor: Thomas E. Wagner, Greenville, SC (US)

(73) Assignee: ORBIS HEALTH SOLUTIONS LLC, Greenville, SC (US)

(21) Appl. No.: 15/751,640

(22) PCT Filed: Aug. 10, 2016
(86) PCT No.: PCT/US16/46308

§ 371 Date: Feb. 9, 2018

Related U.S. Application Data
(60) Provisional application No. 62/303,701, filed on Mar. 4, 2016.

(51) Int. Cl. A61K 39/095; A61K 39/12

(52) U.S. Cl. CPC A61K 39/095; A61K 2039/523; A61K 39/12

(57) ABSTRACT

The present invention generally relates to compositions and methods for delivering a vaccine. The compositions and methods disclosed herein are particularly useful in making bacterial and viral vaccines.

### FIELD OF THE INVENTION

The present invention generally relates to compositions comprising yeast cell wall particles and methods for delivering a vaccine. The compositions and methods disclosed herein are particularly useful in making bacterial and viral vaccines.

### BACKGROUND OF THE INVENTION

According to the World Health Organization, infectious disease remains a leading cause of death, especially in low-income countries. Viral and bacterial infections are a major public health concern. Vaccines that induce protective immunity plays an important role in infectious disease control or elimination.

Conventional vaccines consist of attenuated pathogens, killed pathogens, or immunogenic components of the pathogen. Sub-unit vaccines such as recombinant proteins and synthetic peptides are emerging as novel vaccine candidates. Although some antigens used as sub-unit vaccines are highly immunogenic, a lot of antigens, fail to induce an immune response or induce only a weak immune response. One way to improve immune response of vaccines is by targeted delivery of the immunogenic material to a cell of monocytic origin, such as dendritic cells. Recently, many studies have reported targeted delivery systems for delivering biological materials to dendritic cells. For example, it was reported that microspheres/microparticles, liposomes, nanoparticles, dendrimers, niosomes, and carbon nanotubes could be used for this purpose. Jain et al., Expert Opin. Drug Deliv. 10(3): 353-367 (2013). However, there remains a need in the art to provide more effective vaccines against bacterial and viral pathogens, for example, vaccines with sustained delivery, reduced dose and fewer adverse effects. The present invention provides a novel vaccine composition that satisfies this need.

### SUMMARY OF THE INVENTION

In one aspect, the present invention relates to a vaccine comprising (i) a yeast cell wall particle, and (ii) an antigen loaded within the yeast cell call particle, wherein the vaccine upon administration to a human stimulates an immune response.

In some embodiments, the antigen is selected from the group consisting of viral antigens and bacterial antigens. In some specific embodiments, the antigen is a bacterial antigen. In a preferred embodiment, the antigen is a protein derived from N. Meningitidis, or a fragment thereof. In another preferred embodiment, the antigen is recombinant protein A05 or B01 from N. meningitidis, or a fragment thereof. In another preferred embodiment, the antigen is a combination of recombinant protein A05 or B01 from N. meningitidis.

In some embodiments, the antigen is a viral antigen. In some embodiments, the antigen is a protein derived from influenza A. In a preferred embodiment, the antigen is hemagglutinin of influenza A. In some embodiments, the antigen is a protein derived from HIV. In a preferred embodiment, the antigen is gp120 of HIV.

In some embodiments, the yeast cell wall particle present in the vaccine of the present invention comprises a silicate coated particle. In some embodiments, the yeast cell wall particle is modified by capping with the silicate. In a preferred embodiment, the silicate comprises an organic moiety attached to each of the four oxygen compounds of an orthosilicate. In another preferred embodiment, the silicate is selected from a group consisting of tetraethylorthosilicate, tetramethlorthosilicate, tetrapropylorthosilicate, or tetrabutylorthosilicate. In a preferred embodiment, the silicate is tetraorthosilicate.

In some embodiments, the vaccine of the present invention further comprising one or more adjuvants, excipients and preservatives. Commonly used adjuvants include but are not limited to proteins, peptides, nucleic acids and carbohydrates. Exemplary adjuvants include but are not limited to monophosphoryl lipid A, LPS, CpG oligonucleotides (such as CpG DNA), Poly I:C, Poly ICLC, potent MHC II epitope peptides, beta glucan, and dendritic cell stimulating cytokines such as IL-12 and IFN-γ, as well as DC maturing cytokines such as IL-4 and GM-CSF. Suitable adjuvants are those molecules known to mature DC and interact with receptors on dendritic cells in order to activate dendritic cells and further stimulate a more robust generation of T cells, such as CD4+ and CD8+ T cells. In some embodiments, the adjuvants are loaded within the yeast cell wall particle. In a preferred embodiment, the adjuvant is monophosphoryl lipid A or CpG oligonucleotide.

In another aspect, the present invention provides a method for efficiently delivering a vaccine to a subject comprising administering a vaccine of the present invention. In some embodiments, the vaccine is administered subcutaneously, orally, or intravenously. In some embodiments, the vaccine is directly administered to the dermis of the subject.

### BRIEF DESCRIPTION OF THE DRAWINGS

FIG. 1 depicts antibody titers against protein Neisseria meningitidis Serogroup A05 in mice vaccinated with A05 loaded in yeast cell wall particles or A05 with alum adjuvant (Imject Alum, Thermo scientific). Serum from mice without vaccination was used as control. Results show yeast cell wall particles loaded with recombinant protein A05 induce strong antibody responses with titers higher than 1:2000 dilution, which is stronger than the antibody response induced by recombinant proteins with alum adjuvant.

FIG. 2 depicts antibody titers against protein Neisseria meningitidis Serogroup B01 in mice vaccinated with B01 loaded in yeast cell wall particles or B01 loaded with alum adjuvant (Imject Alum, Thermo scientific). Serum from mice without vaccination was used as control. Results show recombinant protein induces strong antibody responses with titers higher than 1:6000, which is stronger than the antibody response induced by recombinant proteins with alum adjuvant.

FIG. 3 depicts antibody titers against hemagglutinin from influenza virus in mice vaccinated with hemagglutinin in yeast cell wall particles (Imject Alum, Thermo scientific) or hemagglutinin with alum adjuvant. Serum from mice without vaccination was used as control. Results show recombinant protein induces strong antibody responses with titers higher than 1:4000, which is stronger than the antibody response induced by hemagglutinin with alum adjuvant.

### DETAILED DESCRIPTION OF THE PREFERRED EMBODIMENTS

The present invention is not to be limited in terms of the particular embodiments described in this application, which are intended as single illustrations of individual aspects of the invention. All the various embodiments of the present invention will not be described herein. Many modifications and variations of the invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

Reference is made herein to various methodologies known to those of ordinary skill in the art. Publications and other materials setting forth such known methodologies to which reference is made are incorporated herein by reference in their entirety as though set forth in full.

### Definitions

The term “about” in connection with numerical values and ranges means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. For example, “about” means that ⁺/−10% of a particular numerical value following the term.

As used herein, the “administration” of an agent to a subject includes any route of introducing or delivering the agent to a subject to perform its intended function. Administration can be carried out by any suitable route, including intravenously, intramuscularly, intraperitoneally, or subcutaneously. Administration can also be carried out by injection to the dermis of the subject. Administration includes self-administration and the administration by another.

As used herein “subject” or “patient” denotes any animal in need of treatment with a vaccine. For example, a subject may be suffering from or at risk of developing a condition that can be treated or prevented with a vaccine. As used herein “subject” or “patient” includes humans.

The term “comprising” is intended to mean that the compositions and methods described herein include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the phrases “therapeutically effective amount” and “therapeutic level” mean that the vaccine dosage or plasma concentration of the compositions described herein in a subject, respectively, that provides the specific response for which the biological material or vaccine is administered in a subject in need of such treatment. For convenience only, exemplary dosages, delivery amounts, therapeutically effective amounts and therapeutic levels are provided below with reference to adult human subject. Those skilled in the art can adjust such amounts in accordance with standard practices as needed to treat a specific subject and/or condition/disease.

As used herein, the term “protein” means a polypeptide (native [i.e., naturally-occurring] or mutant), peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic including peptides with nitrogen based backbone. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

As used herein, the term “recombinant” as used herein means that a protein or polypeptide employed in the invention is derived from recombinant (e.g., microbial or mammalian) expression systems. “Microbial” refers to recombinant proteins or polypeptides made in bacterial or fungal (e.g., yeast) expression systems. As a product, “recombinant microbial” defines a protein or polypeptide produced in a microbial expression system, which is essentially free of native endogenous substances. Proteins or polypeptides expressed in most bacterial cultures, e.g. E. coli, will be free of glycan. Proteins or polypeptides expressed in yeast may have a glycosylation pattern different from that expressed in mammalian cells.

For purposes of this invention, “homology” or “homologous” refers to the percent homology between two polynucleotide moieties or two polypeptide moieties. The correspondence between the sequence from one moiety to another can be determined by techniques known in the art. Two DNA or two polypeptide sequences are “substantially homologous” to each other when at least about 80%, preferably at least about 90%, and most preferably at least about 95% of the nucleotides or amino acids match over a defined length of the molecules, as determined using methods in the art.

The techniques for determining amino acid sequence homology are well-known in the art. In general, “homology” (for amino acid sequences) means the exact amino acid to amino acid comparison of two or more polypeptides at the appropriate place, where amino acids are identical or possess similar chemical and/or physical properties such as charge or hydrophobicity. A so-termed “percent homology” then can be determined between the compared polypeptide sequences. The programs available in the Wisconsin Sequence Analysis Package (available from Genetics Computer Group, Madison, Wis.), for example, the GAP program, are capable of calculating homologies between two polypeptide sequences. In addition, the ClustalW algorithm is capable of performing a similar analysis. Other programs and algorithms for determining homology between polypeptide sequences are known in the art.

As used herein, the term “antigen” or “immunogen” means a substance that induces a specific immune response in a host animal. The antigen may comprise a whole organism, killed, attenuated or live; a portion of an organism; a recombinant vector containing an insert with immunogenic properties; a piece or fragment of DNA capable of inducing an immune response upon presentation to a host animal; a polypeptide, an epitope, a hapten, or any combination thereof. Alternately, the immunogen or antigen may comprise a toxin or antitoxin.

As used herein, the term “immunogenic or antigenic polypeptide” as used herein includes polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques well known in the art. See, e.g., Epitope Mapping Protocols in Methods in Molecular Biology, Vol. 66 (Glenn E. Morris, Ed., 1996). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. An antigen used in the present invention can be a viral antigen, a parasite antigen, and/or a bacterial antigen. The antigen of the present invention does not cause illness but can effectively provoke an immune response of the subject and protects the subject against future infection of a particular disease, or minimize the severity of a particular condition.

As used herein, the term “particle” refers to any hollow and porous structure that can encapsulate an agent therein and also allow the agent to exit the structure. A particle used in the present invention may include particles of any shape, for example, spherical, tube or rod, provided the pore structure is suitable for receiving the encapsulated agent. The particle may have a rough surface, smooth surface, angular surfaces, or sharp edges, or may have a regular or irregular shape. The particle material may comprise any biocompatible and biodegradable material.

As used herein, the term “immunological response” or “immune response” as used herein can include the development in the subject of a humoral and/or a cellular immune response to the antigen used when the antigen is present in a vaccine composition. Antibodies elicited in an immune response may also neutralize infectivity, and/or mediate antibody-complement or antibody dependent cell cytotoxicity to provide protection to an immunized host. Immunological reactivity may be determined in standard immunoassays, such as a competition assays, well known in the art. The immunoassays suitable for use depend on the specific antigen in a vaccine of the present invention.

As used herein, the term “capping” or “capped” means a thin polymeric structure over the outside of the porous shell of the cavity of the empty particle in the present invention that serves to slow or to prevent the release of an encapsulated agent from the hollow inside of the particle used in the present invention, such as yeast cell wall particle. Thus, as used herein, “capping” or “capped” includes partially or fully blocking the opening of a pore such that the release of an encapsulated agent is slowed or prevented. A cap can comprise a variety of materials, which can be selected based on the intended application for the loaded particles and on the size and reactivity of the particle material. A capped yeast cell wall particle comprises a polymeric structure, like a “mesh net,” covers or coats the yeast cell wall particle such that the biological material loaded within the yeast cell wall particle is retained or entrapped therein. The polymeric structure can be formed by a silicate, such as a orthosilicate.

Orthosilicates useful in the compositions and methods described herein are represented by the following formula: Si(OR)₄, wherein R is a C₁-C₁₂ alkyl. For example, R can be a methyl group, an ethyl group, a propyl group, an butyl group, a pentyl group, a hexyl group, an heptyl group, an octyl group, a nonyl group, a decyl group, an undecyl group, a dodecyl group. In a preferred embodiment, the orthosilicate in the present invention is tetraorthosilicate.

The term “excipient” means diluents or other components used in the formulation of the vaccine composition. Excipients can include: diluents or fillers, binders or adhesives, dissolution aids, lubricants, antiadherents, glidants or flow promoters, colors, flavors, sweeteners and adsorbents.

The term “preservative” means a compound that can be added to the diluent to essentially reduce bacterial action in the reconstituted formulation, thus facilitating the production of a multi-use reconstituted formulation, for example. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, 2-phenoxyethanol, thimerosal, benzethonium chloride, formaldehyde, butyl and benzyl alcohol, allyl parabens such as methyl or propyl paraben, catechol, resorcinol, cyclohexanol, 3-pentanol, and m-cresol. The most preferred preservative herein is 2-phenoxyethanol.

The term “immunization” means a process by which a subject becomes protected against a particular condition, disease or diseases, usually by receiving a vaccine.

The term “vaccine” is a biological material or product that induces an immune response in the body of a subject upon administration, e.g., by injection, by oral administration, or by aerosol administration. The vaccine comprises at least one active component, such as an antigen that induces immune response, and at least one additional component such as an adjuvant, a preservative, or other excipient including a diluent, a stabilizer, etc.

### Vaccine

The vaccine of the present invention comprises an agent encapsulated in a particle. The agent encompassed by this invention includes, but is not limited to, an antigen, such as a specific protein or a fragment thereof, nucleic acid, carbohydrate, protein, peptide, or a combination thereof. One of ordinary skill in the art would understand that fragments of a protein, e.g. a peptide of any length, an epitope, or a subunit of a protein, which produce immunogenic response of a subject upon administration can be used.

Nucleic acids such as DNA, RNA, cDNA or fragments thereof, are also used as an agent. In general, the DNA is extracted from an infectious agent's DNA, and then modified/enhanced by genetic engineering before delivering to a subject by electroporation, gene gun, etc.

The antigen of the present invention may be live, wild-type pathogens, or in inactivated or attenuated forms, such as killed viruses, pieces of bacteria, and subunits or immunogenic functional fragments of proteins, polypeptides or nucleic acids. More preferably, the antigen does not cause illness but can effectively provoke an immune response of the subject and protects the subject against future infection of a particular disease, or minimize the severity of a particular condition.

It is to be understood that yeast cell wall particles have a pore size of at least about 30 nm, and therefore, any molecule/object with a radius of 30 nm or less can be loaded within the yeast cell wall particles. For example, some viruses or viral particles having a size less than 30 nm (e.g., tobacco mosaic virus) can be loaded within the yeast cell wall particles, as well as other antigens, including bacterial antigens.

### Bacterial Antigen

In some embodiments, the vaccine of the present invention comprises a bacterial antigen. A bacterial antigen encompasses all substances that are capable of eliciting an immune response against a bacterium, for example, inactivated or attenuated bacteria, pieces of bacteria, and subunits or immunogenic functional fragments of proteins or polypeptides derived from bacteria.

In some embodiments, the bacterial antigen is derived from Helicobacter pyloris; Borelia species, in particular Borelia burgdorferi; Legionella species, in particular Legionella pneumophilia; Mycobacteria species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumoniae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella pneumoniae, Pasteurella species, in particular Pasteurella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israelli. The bacterial antigen or a fragment thereof, is preferably loaded into the yeast cell wall particles of the present invention, which is capable of stimulating an immune response.

Neisseria meningitidis

In some embodiments, the bacterial antigen is derived from Neisseria meningitidis. Neisseria meningitidis is a gram negative spherical bacterium that can cause meningitis and other forms of meningococcal disease such as meningococcemia, a life-threatening sepsis. N. meningitidis can be classified into about 13 serogroups based on chemically and antigenically distinctive polysaccharide capsules. Five of the serogroups (A, B, C, Y, and W135) are responsible for the majority of disease. The present invention is not limited by the serogroup of N. meningitidis used or immunogenic protein derived therefrom.

In some embodiments, the bacterial antigen derived from Neisseria meningitidis is a protein identified as ORF2086 protein, immunogenic portions thereof, and/or biological equivalents thereof. The term “ORF2086” as used herein refers to Open Reading Frame 2086 from a Neisseria species bacteria. Neisseria ORF2086, the proteins encoded therefrom, fragments of those proteins, and immunogenic compositions comprising those proteins are known in the art and are described, e.g., in U.S. Patent Application Publication Nos. US 20060257413 and US 20090202593, each of which are hereby incorporated by reference in their entirety. The term “P2086” generally refers to the protein encoded by ORF2086. The P2086 proteins of the invention may be lipidated or non-lipidated. “LP2086” and “P2086” typically refer to lipidated and non-lipidated forms of a 2086 protein, respectively. LP2086 can be divided into two serologically distinct subfamilies (A and B). The present invention is not limited by the subfamilies of N. meningitidis used or immunogenic protein derived therefrom.

In some embodiments, the bacterial antigen further includes other Neisseria species immunogenic peptides, proteins, or fragment thereof. In some embodiment, the bacterial antigen may include a combination of two or more ORF2086 proteins, a combination of ORF2086 protein with one or more proteins from N. meningitidis serogroup A, C, Y and W135, or polysaccharides and/or polysaccharide conjugates from meningococcus serogroup A, C, Y and W135, or a combination of any of the foregoing in a form suitable for a desired administration, e.g., for mucosal delivery. Persons of skill in the art would be readily able to formulate such multi-antigen compositions.

In a preferred embodiment, the bacterial antigen is a LP2086 subfamily A protein or a LP2086 subfamily B protein, or an immunogenic portions thereof. In some embodiments, the bacterial antigen is a A05 variant of the LP2086 subfamily A protein. In other embodiments, the bacterial antigen is a B01 variant of the LP2086 subfamily B protein. In a preferred embodiment, the bacterial antigen comprises a mixture with 1:1 ratio of a subfamily A protein to a subfamily B protein. In another preferred embodiment, the bacterial antigen comprises a mixture of equal amount of A05 variant and B01 variant of LP2086 subfamily A proteins.

In some embodiment, a variant of a LP2086 subfamily A protein or a LP2086 subfamily B protein can be used as antigen in the vaccine of the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against N. meningitidis. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the LP2086 subfamily A protein or the LP2086 subfamily B protein disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the LP2086 subfamily A protein or the LP2086 subfamily B protein, or to increase or decrease the immunogenicity, solubility or stability of the proteins described herein.

Methods for producing LP2086 proteins and their variants are known in the art. See U.S. Pat. No. 8,568,743. The present invention contemplates any changes to the structure of the polypeptides herein, as well as the nucleic acid sequences encoding said polypeptides, wherein the polypeptide retains immunogenicity.

It is also contemplated by the present invention that a LP2086 subfamily A protein or subfamily B protein from N. meningitides can be cleaved into fragments for use in a vaccine, wherein the fragments still have N. meningitides immunogencity. This can be accomplished by treating purified or unpurified N. meningitidis proteins with a peptidase such as endoproteinase glu-C (Boehringer, Indianapolis, Ind.). Treatment with CNBr is another method by which peptide fragments may be produced from natural N. meningitidis 2086 polypeptides. See U.S. Pat. No. 8,568,743.

The LP2086 protein and a fragment thereof can be prepared recombinantly, as is well known within the skill in the art, based upon the guidance provided herein, or in any other synthetic manner, as known in the art. The sequences of LP2086 A05 and B01 proteins are shown below.

LP2086 A05_001 Amino Acid sequence
(SEQ ID NO: 1)
MCSSGSGSGGGGVAADIGTGLADALTAPLDHKDKGLKSLTLEDSISQ
NGTLTLSAQGAEKTFKVGDKDNSLNTGKLKNDKISRFDFVQKIEVDG
QTITLASGEFQIYKQDHSAVVALQIEKINNPDKIDSLINQRSFLVSG
LGGEHTAFNQLPSGKAEYHGKAFSSDDAGGKLTYTIDFAAKQGHGKI
EHLKTPEQNVELASAELKADEKSHAVILGDTRYGSEEKGTYHLALFG
DRAQEIAGSATVKIREKVHEIGIAGKQ(HHHHHH)
(extra 6xhis tag)
LP2086 B01_001 Amino Acid sequence
(SEQ ID NO: 2)
MCSSGGGGSGGGGVTADIGTGLADALTAPLDHKDKGLKSLTLEDSIS
QNGTLTLSAQGAEKTYGNGDSLNTGKLKNDKVSRFDFIRQIEVDGQL
ITLESGEFQVYKQSHSALTALQTEQEQDPEHSEKMVAKRRFRIGDIA
GEHTSFDKLPKDVMATYRGTAFGSDDAGGKLTYTIDFAAKQGHGKIE
HLKSPELNVDLAVAYIKPDEKHHAVISGSVLYNQDEKGSYSLGIFGE
KAQEVAGSAEVETANGIHHIGLAAKQ(HHHHHH)
(extra 6xhis tag)

The immunogenicity of a LP2086 subfamily A protein, a LP2086 subfamily B protein, or a variant thereof, can be measured as the ability of a vaccine comprising such proteins to elicit bactericidal antibody titers against N. meningitides. Methods of determining antibody titers and methods of performing antibody titer assays are also known to those skilled in the art. See Fletcher et al., Infection & Immunity. 72(4):2088-2100 (2004). Other methods commonly known by those skilled in the art may also be used to measure the immunogenicity of a vaccine against N. meningitidis.

### Viral Antigens

In some embodiments, the vaccine of the present invention comprises a viral antigen. A viral antigen comprises all substances capable of eliciting an immune response against a virus, an inactivated or attenuated viruses, pieces of viruses, and subunits or immunogenic functional fragments of proteins or polypeptides derived from viruses.

### Influenza

In some embodiments, the present invention comprises a viral antigen derived from an influenza virus. Influenza viruses can be classified into types A, B and C. The present invention is not limited by the type of influenza virus used or immunogenic protein derived therefrom. In some embodiments, the vaccine of the present invention is for Influenza type A viruses. Influenza type A viruses may be further divided into subtypes according to the combination of hemagglutinin (HA) and neuraminidase (NA) surface glycoproteins presented on viruses. Currently, 16 HA (H1-H16) subtypes and 9 NA (N1-N9) subtypes are recognized. Each type A influenza virus presents one type of HA and one type of NA glycoprotein. In some embodiments, the present invention contemplates vaccines against influenza virus subtype H1N1, H2N2, H3N2, H5N1, H8N2, H7N7, and H7N9, which have been isolated from human.

In some embodiments, the viral antigen is influenza hemagglutinin protein, a membrane glycoprotein from influenza virus. The hemagglutinin polypeptide may be derived from any influenza virus type, subtype, strain or substrain, such as from the H1, H2, H3, H5, H7 and H9 hemagglutinins. In some embodiments, the viral antigen used in the present invention comprises an amino acid sequence capable of eliciting an immune response and derived from hemagglutinin protein of an influenza virus selected from A/New Caledonia/20/1999 (1999 NC, HI), A/California/04/2009 (2009 CA, HI), A/Singapore/1/1957 (1957 Sing, H2), A/Hong Kong/1/1968 (1968 HK, H3), A/Brisbane/10/2007 (2007 Bris, H3), A/Indonesia/05/2005 (2005 Indo, H5), B/Florida/4/2006 (2006 Flo, B), A/Perth/16/2009 (2009 Per, H3), A/Brisbane/59/2007 (2007 Bris, HI), B/Brisbane/60/2008 (2008 Bris, B). In addition, the hemagglutinin polypeptide may be a chimera of different influenza hemagglutinins. In a preferred embodiment, the viral antigen comprises the amino acid sequence or a fragment of hemagglutinin from Influenza A virus subtype H5N1 (A/Hong kong/483/97), the sequence of which is shown below.

Hemagglutinin from Influenza A virus
(A/Hong Kong/483/97 (H5N1)
(SEQ ID NO: 3)
MEKIVLLLAT VSLVKSDQIC IGYHANNSTE QVDTIMEKNV
TVTHAQDILE RTHNGKLCDL NGVKPLILRD CSVAGWLLGN
PMCDEFINVP EWSYIVEKAS PANDLCYPGN FNDYEELKHL
LSRINHFEKI QIIPKSSWSN HDASSGVSSA CPYLGKSSFF
RNVVWLIKKN STYPTIKRSY NNTNQEDLLV LWGIHHPNDA
AEQTKLYQNP TTYISVGTST LNQRLVPEIA TRPKVNGQSG
RIEFFWTILK PNDAINFESN GNFIAPEYAY KIVKKGDSTI
MKSELEYGNC NTKCQTPMGA INSSMPFHNI HPLTIGECPK
YVKSNRLVLA TGLRNAPQRE RRRKKRGLFG AIAGFIEGGW
QGMVDGWYGY HHSNEQGSGY AADQESTQKA IDGVTNKVNS
IINKMNTQFE AVGREFNNLE RRIENLNKKM EDGFLDVWTY
NAELLVLMEN ERTLDFHDSN VKNLYDKVRL QLRDNAKELG
NGCFEFYHKC DNECMESVKN GTYDYPQYSE EARLNREEIS
GVKLESMGTY QILSLYSTVA SSLALAIMVA GLSLW

The hemagglutinin used in the vaccine of the present invention can be prepared from influenza virions or expressed in a recombinant host (e.g. in an insect cell line using a baculovirus vector) and used in purified form. The method of producing hemagglutinin is well known in the art. See Andrianov et al. Biomaterials 19:109-115 (1998), Banzhoff Immunology Letters 71:91-96 (2000), and Beignon et al. Infect Immun. 70:3012-3019 (2002).

In some embodiments, a variant of hemagglutinin can be used as antigen in the vaccine of the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against an influenza virus. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the influenza HA proteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the HA protein, or to increase or decrease the immunogenicity, solubility or stability of the HA proteins described herein.

Methods for producing hemagglutinin protein and its variants are known in the art. See Wei et al., J Virol 82: 6200-6208 (2008), and Wei et al., Science 329: 1060-1064 (2010). The immunogenicity of a vaccine comprising an antigen derived from hemagglutanin from influenza A virus may be measured as the ability of a protein to activate T cell response. Methods of determining T cell response to antigen, for example, using mixed lymphocyte reaction are also known to those skilled in the art. See Mason, et al. Immunology, 44(1):75-87 (1981), and Steinman, et al. U.S. Pat. No. 6,300,090. Other methods commonly known by those skilled in the art may also be used to measure the immunogenicity of a vaccine against influenza virus.

### HIV

In some embodiments, the viral antigen of the present invention is derived from human immunodeficiency virus (HIV). The present invention is not limited by the type of HIV used or immunogenic protein derived therefrom. For example, two types of HIV, HIV-1 and HIV-2 are both contemplated. In some embodiments, the viral antigen is an immunogenic protein selected from HIV glycoproteins (gp120, gp160, and gp41), HIV nonstructural proteins (Rev, Tat, Nif and Nef), or a combination thereof. In some embodiments, the viral antigen can be fragments of the full length HIV proteins or fragments that are capable of eliciting an immune response. In a preferred embodiment, the viral antigen is gp120 of HIV-1.

Furthermore, the HIV glycoprotein is not limited to a polypeptide having the exact sequence described herein. Indeed, the HIV genome is in a state of constant flux and contains several variable domains that exhibit relatively high degrees of variability between isolates. It is readily apparent that the HIV glycoprotein of the present invention encompasses polypeptides from any of the identified HIV isolates, as well as newly identified isolates, and subtypes of these isolates. One of ordinary skill in the art in view of the teachings of the present disclosure and the art can determine corresponding regions in other HIV variants (e.g., isolates HIV_{IIIb}, HIV_{SF2}, HIV-1_{SF162}, HIV-1_{SF170}, HIV_{LAV}, HIV_{LAI}, HIV_{MN}, HIV-1_{CM4235}, HIV-1_{US4}, other HIV-1 strains from diverse subtypes (e.g., subtypes, A through G, and O), HIV-2 strains and diverse subtypes (e.g., HIV-2_{UC1} and HIV-2_{UC2}), and simian immunodeficiency virus (SIV), using for example, sequence comparison programs (e.g., BLAST and others described herein) or identification and alignment of structural features (e.g., a program such as the “ALB” program described herein that can identify n-sheet regions). See, e.g., Virology, 3rd Edition (W. K. Joklik ed. 1988); Fundamental Virology, 2nd Edition (B. N. Fields and D. M. Knipe, eds. 1991); Virology, 3rd Edition (Fields, B N, D M Knipe, P M Howley, Editors, 1996, Lippincott-Raven, Philadelphia, Pa.).

In some embodiments, a variant of HIV glycoproteins can be used in the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against HIV. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the HIV glycoproteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the HIV glycoprotein, or to increase or decrease the immunogenicity, solubility or stability of the HIV glycoproteins described herein.

Methods of producing HIV glycoproteins and the variants thereof are known in the art. For example, various cloning vectors and expression systems are described in generally, DNA Cloning: Vols. I & II; U.S. Pat. No. 5,340,740; Yeast Genetic Engineering (Barr et al., eds., 1989) Butterworths; Tomei et al., J. Virol. 67:4017-4026 (1993); and Selby et al., J. Gen. Virol. 74:1103-1113 (1993).

The immunogenicity of a vaccine comprising an antigen derived from a HIV glycoprotein may be measured as the ability of a protein to activate T cell response. Methods of determining T cell response to antigen, for example, using mixed lymphocyte reaction is also known to those skilled in the art. See U.S. Pat. No. 7,566,568. Other methods commonly known by those skilled in the art may also be used to measure the immunogenicity of a vaccine against HIV.

### HPV

In some embodiments, the viral antigen of the present invention is derived from human papillomavirus (HPV). The present invention is not limited by the type of HPV used or immunogenic protein derived therefrom. For example, the viral antigen may be derived from HPV-1, HPV-2, HPV-5, HPV-6, HPV-11, HPV-18, HPV-31, HPV-45, HPV-52, and HPV-58, bovine papillomavirus-1, bovine papillomavirus-2, bovine papillomavirus-4, cottontail rabbit papillomavirs, or rhesus macaque papillomavirus. In some embodiments, the viral antigen comprises the amino acid sequence from papillomavirus capsid protein L1 and/or L2. In some embodiments, the viral antigen comprises amino acid sequence from papillomavirus early antigen proteins E1, E2, E3, E4, E5, E6, and E7, or a combination thereof. In some embodiments, the viral antigen comprises one or more fragments of the HPV proteins mentioned above that are capable of eliciting an immune response.

In some embodiments, the vaccine compositions of the present invention comprise HPV L1 or HPV L2, or L1+L2 proteins of at least one type of HPV. HPV L1, HPV L2, or HPV L1+L2 protein can be expressed recombinantly by molecular cloning of L1, L2, or L1+L2 DNA into an expression vector containing a suitable promoter and other appropriate transcription regulatory elements, and transferred into prokaryotic or eukaryotic host cells to produce recombinant protein. Techniques for such manipulations are fully described by Sambrook et al. (Molecular Cloning: A Laboratory Manual; Cold Spring Harbor Laboratory, Cold Spring Harbor, N.Y., (1989)), which is hereby incorporated by reference.

In some embodiments, a variant of HPV L1 or L2 protein can be used in the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against HPV. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the HPV L1 or L2 protein disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the HPV L1 or L2 protein, or to increase or decrease the immunogenicity, solubility or stability of the HPV L1 or L2 protein described herein.

Methods of producing HPV L1 and L2 proteins and the variants thereof are known in the art. See, e.g., U.S. Pat. No. 5,820,870 and Kirii et al. Virology 185(1): 424-427 (1991).

The immunogenicity of a vaccine comprising a HPV antigen can be measured, for example, by neutralization antibody binding assay (Surface Plasmon Resonance, Biacore). The Biacore conditions utilized were as described in Mach et al. J. Pharm. Sci. 95: 2195-2206 (2006). Other methods commonly known by those skilled in the art may also be used to measure the immunogenicity of a vaccine against HPV.

### Herpes Simplex Virus

In some embodiments, the viral antigen of the present invention is derived from herpes simplex virus (HSV), for example, from HSV-1 or HSV-2. The present invention is not limited by the type of HSV used or immunogenic protein derived therefrom. Any known HSV strain can be used in the vaccines of the present invention. Examples of useful strains of HSV include, but are not limited to, HSV strain deposited with the ATCC, such as: (1) HSV Strain HF (ATCC VR-260; Human herpesvirus 1); (2) HSV Strain MacIntyre (ATCC VR-539; Human herpesvirus 1); (3) HSV Strain MS (ATCC VR-540; Human herpesvirus 2); (4) HSV Strain F (ATCC VR-733; Human herpesvirus 1); (5) HSV Strain G (ATCC VR-734; Human herpesvirus 2); (6) HSV Strain MP (ATCC VR-735; Human herpesvirus 1, mutant strain of herpes simplex virus type 1); (7) Mutant Strain of HSV (ATCC VR-1383; Human herpesvirus 1, mutant strain of herpes simplex virus type 1); (8) HSV Stain KOS (ATCC VR-1493; Human herpesvirus 1; derived from ATCC VR-1487 by passage in the presence of MRA to remove mycoplasma contaminants); (9) HSV Strain ATCC-201 1-1 (ATCC VR-1778; Human herpesvirus 1); (10) HSV Strain ATCC-201 1-2 (ATCC VR-1779; Human herpesvirus 2); (1 1) HSV Strain ATCC-201 1-4 (ATCC VR-1781; Human herpesvirus 2); (12) HSV Strain A5C (ATCC VR-2019; Human herpesvirus 1×2 (recombinant); Source: Crossing of parental strains of HSV-1 (17ts) and HSV-2 (GPG)); (13) HSV Strain D4E3 (ATCC VR-2021; Human herpesvirus 1×2 (recombinant); Source: Crossing of parental strains of HSV-1 (KOStsE6) and HSV-2 (186tsB5)); (14) HSV Strain C7D (ATCC VR-2022; Human herpesvirus 1×2 (recombinant); Source: Crossing of parental strains of HSV-1 (HFEMtsN102) and HSV-2 (186)); (15) HSV Strain D3E2 (ATCC VR-2023; Human herpesvirus 1×2 (recombinant); Source: Crossing of parental strains of HSV-1 (KOStsE6) and HSV-2 (186tsB5)); (16) HSV Strain C5D (ATCC VR-2024; Human herpesvirus 1×2 (recombinant); Source: Crossing of parental strains of HSV-1 (HFEMtsN102) and HSV-2 (186); (17) HSV Strain D5E1 (ATCC VR-2025; Human herpesvirus 1×2 (recombinant); Source: Crossing of parental strains of HSV-1 (KOStsE6) and HSV-2 (186tsB5); and (18) HSV Strain D1 E1 (ATCC VR-2026; Human herpesvirus 1×2 (recombinant); Source: Crossing of parental strains of HSV-1 (KOStsE6) and HSV-2 (186tsB5)).

Additionally, in some embodiments, the viral antigen present in the vaccine of the invention comprises the amino acid sequence from herpes simplex virus antigen gB, gC, gD, or gE, or a combination thereof. References to amino acids of HSV proteins or polypeptides are based on the genomic sequence information as described in McGeoch et al., J. Gen. Virol. 69:1531-1574 (1988). In some embodiments, the HSV antigen comprises one or more fragment of these proteins. The HSV antigens are generally extracted from viral isolates from infected cell cultures, or produced by synthetically or using recombinant DNA methods. The HSV surface antigens can be modified by chemical, genetic or enzymatic means resulting in fusion proteins, peptides, or fragments. See, U.S. Pat. No. 6,375,952. The HSV surface antigens can be obtained from any known HSV strain, including but not limited to the strains listed above.

A HSV antigen of the present invention may also comprise fragments of HSV gB, gC, gD, or gE protein, for example deletion mutants, truncation mutants, oligonucleotides, and peptide fragments, provided that the fragments are immunogenic. In some embodiments, the present invention may comprise multiple fragments derived from at least one of HSV HSV gB, gC, gD, and gE proteins. As is understood in the art and confirmed by assays conducted using fragments of widely varying lengths, a fragment of the invention can encompass 10%, 20%, 30%, 40%, 50%, 60%, 70%, 80%, 90% or 95% of the full length of the protein, provided the fragments are capable of eliciting an immune response.

In some embodiment, a variant of HSV glycoprotein can be used in the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against HSV. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the HSV glycoproteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the HSV glycoprotein, or to increase or decrease the immunogenicity, solubility or stability of the HSV glycoproteins described herein.

Fragments and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, wherein amino acids are sequentially added to a growing amino acid chain. See, Merrifield, J. Am. Chem. Soc. 85:2146-2149 (1963). Equipment for automated synthesis of polypeptides is commercially available from suppliers such as Perkin Elmer/Applied BioSystems Division (Foster City, Calif.), and may be operated according to the manufacturer's instructions.

Methods of producing HSV glycoproteins and the variants thereof are known in the art. See, U.S. Pat. No. 8,617,564. The immunogenicity of a vaccine comprising a HSV antigen can be measured by various methods, including protein microarray and ELISPOT/ELISA technique. See, U.S. Pat. No. 8,617,564. Briefly, a series of dilutions of the antibody which binds to the antigen or the antigen variant is made in replicate samples. The binding affinity of the antigen for the antibody is then measured for the different concentrations. The same process is then carried out with the antigen replaced by the antigenic variant. A two-way analysis of variance (i.e. a statistical test) is carried out to assess the significance of differences between the results for the antigen and the antigenic variant. Other methods commonly known by those skilled in the art may also be used to measure the immunogenicity of a vaccine against HSV.

### Poxvirus

In some embodiments, the viral antigen of the present invention is derived from poxvirus. The present invention is not limited by the type of poxvirus used or immunogenic protein derived therefrom. Any known poxvirus strain can be used in the vaccines of the present invention. Examples of useful strains of poxvirus include, but are not limited to smallpox virus, cowpox virus, buffalopox virus, camelpox virus, ectromelia virus, elephantpox virus, horsepox virus, monkeypox virus, rabbitpox virus, raccoonpox virus, skunkpox virus, tatera poxvirus, Uasin Gishu disease virus, volepox virus, vaccinia virus and variola virus, of which, the camelpox virus is more preferably of the strain camelpox virus 903, camelpox virus CMG, camelpox virus CMS, camelpox virus CPI, camelpox virus CP5, camelpox virus M-96, the cowpox virus is more preferably of the strain Brighton Red, strain GRI-90, Hamburg-1985 or Turkmenia-1974, the ectromelia virus is more preferably of the strain belo horizonte virus or Moscow strain, the monkeypox virus is more preferably of the strain Callithrix jacchus orthopoxvirus, Sierra Leone 70-0266, Zaire-77-0666, the rabbitpox virus is more preferably of the Utrecht strain, the vaccinia virus is more preferably of the strain Ankara, Copenhagen, Dairen I, IHD-J, L-IPV, LC16M8, LC1 6MO, Lister, LIVP, Tashkent, Tian Tan, WR 65-16, WR, Wyeth and the variola virus is more preferably a variola major virus or variola minor virus, or is a antigenic analog thereof.

In some embodiments, the viral antigen comprises the amino acid sequence of poxvirus protein IMV or poxvirus protein EEV derived from any strain stated above. The intracellular mature virus (IMV) is efficient at attaching to and infecting cells whilst the extracellular (EEV) form of virus is actively secreted from cells and contributes to the efficient dissemination of virus in vitro and in vivo. In some embodiments, the viral antigen is a IMV antigen selected from the group consisting of L1R, A27L, A3L, A10L, A12L, A13L, A14L, A17L, D8L, H3L, L4R, G7L, and 15L. In some embodiments, the viral antigen is a EEV antigen selected from the group consisting of A33R, A34R, A36R, A56R, B5R, and F13L. The sequence of the proteins are described in Parkinson, et. al, Virology, 204: 376-90 (1994), and Salmons, et al. Virology, 71:7404-7420 (1997), and US Patent Application 2010/0119524. In some embodiments, the viral antigen comprises a combination of any of these proteins. In some embodiment, the viral antigen comprises a fragment of any of these proteins, provided that the fragment can elicit an immune response.

In some embodiments, a variant of a poxvirus antigen can be used in the present invention. A variant refers to a protein having a sequence that is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety.

With regard to variants, any type of alteration in the amino acid, or nucleic acid, sequence is permissible so long as the resulting variant protein retains the ability to elicit an immune response against poxvirus. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein. As noted, variant proteins of the present invention can contain amino acid substitutions relative to the poxvirus antigen disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Desired amino acid substitutions (whether conservative or non-conservative) can be determined by those skilled in the art at the time such substitutions are desired. For example, amino acid substitutions can be used to identify important residues of the poxvirus antigen, or to increase or decrease the immunogenicity, solubility or stability of the poxvirus antigen described herein.

Methods for producing Poxvirus antigens and their varients are known to persons skilled in the art. See, US Patent Application 2010/0119524. The immunogenicity of a vaccine comprising an poxvirus antigen can be measured by various methods, including ELISA. See US Patent Application Publication No. 2010/0119524. Briefly, a series of dilutions of the antibody which binds to the antigen or the antigen variant is made in replicate samples. The binding affinity of the antigen for the antibody is then measured for the different concentrations. The same process is then carried out with the antigen replaced by the antigen variant. A two-way analysis of variance (i.e. a statistical test) is carried out to assess the significance of differences between the results for the antigen and the antigen variant. Other methods commonly known by those skilled in the art may also be used to measure the immunogenicity of a vaccine against poxvirus.

The antigens contemplated by the present invention is summarized in Table 1.

| | Name | |
|---|---|---|
| | of Species | Antigen |
| Bacteria | N. meningitidis | A05 and B01 variants |
| Virus | HIV | gp120, gp160, gp41, Rev, Tat, Nif and Nef |
| | HPV | L1, L2, E1, E2, E3, E4, E5, E6, and E7 |
| | HSV | gB, gC, gD, or gE |
| | Poxvirus | IMV antigens including L1R, A27L, A3L, |
| | | A10L, A12L, A13L, A14L, A17L, D8L, H3L, |
| | | L4R, G7L, and 15L; EEV antigens including |
| | | A33R, A34R, A36R, A56R, B5R, and F13L |

### Particle

As described herein, “particle” refers to any hollow and porous structure that can contain an agent therein and also allow the agent to exit the structure. The particle of the present invention may have a rough surface, smooth surface, angular surfaces, or sharp edges, and may have a regular or irregular shape. Exemplary shapes of the particle include, but are not limited to, microspheres, rods and tubes. The particle material can comprise any of a wide range of particles, including such exemplary materials as described in U.S. Pat. No. 5,407,609. Biocompatible materials are preferred for uses that involve administration to patients. Biodegradable materials are also preferred, for example poly(lacto-co-glycolide) (PLG), poly(lactide), poly(glycolide), poly(caprolactone), poly(hydroxybutyrate) and/or copolymers thereof. Alternatively, the particle can comprise another material. Suitable other materials include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(²-vinyl pyridine), poly(5-methyl-2-vinyl pyridine) and the like; poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers. In preferred embodiments, the material of the particles have a hollow or porous structure that allows the particle to be phagocytosed by monocytes, including dendritic cells.

In some embodiments, the size of particles of the present invention is 1-25 μm, preferably 1-5 μm, 5-10 μm, 10-15 μm, 15-20 μm, 15-25 μm, or 20-25 μm. In some embodiments, the size of the particle of the present invention is about 0.5 to about 5 μm, which approximates the size of bacterium to allow the particle to be ingested by monocytes, such as dendritic cells. In specific embodiments, the size of the particle is about 0.5 to about 1 μm. In specific embodiments, the size of the particle is about 0.5 to about 2.5 μm. In some embodiments, the particle can be any particle with a glycan network, so long as the particle is about 0.5 to about 5 μm in size.

### Yeast Cell Wall Particles

In a preferred embodiment, the particle of the present invention is a yeast cell wall particle YCWP, which is prepared from yeast cell wall such that the particle has a hollow or porous structure to encapsulate a antigen therein. In one embodiment, the YCWP is prepared from Saccharomyces cerevisiae. In another embodiment, the YCWP approximates the size of microbial structures that cells of the mononuclear phagocyte system and other phagocytic cells typically ingest. In specific embodiments, the YCWP is about 1-25 μm, preferably 1-5 μm, 5-10 μm, 10-15 μm, 15-20 μm, 15-25 μm, or 20-25 μm. For example, the YCWP is about 20 μm.

In one embodiment, the YCWP is prepared by (a) suspending yeast to produce a suspension, (b) incubating the suspension, (c) centrifuging the suspension and removing the supernatant and (d) recovering the resulting YCWP. In another embodiment, steps (a)-(d) are repeated at least 1, 2, 3 or 4 times.

In another embodiment, the YCWP is prepared by (a) suspending yeast in a solution to produce a first suspension, (b) incubating the first suspension, (c) centrifuging the first suspension and removing the supernatant, (d) suspending the resulting pellet to produce a second suspension, (e) incubating the second suspension, (f) centrifuging the second suspension and removing the supernatant and (g) washing the resulting pellet to recover the YCWP. In another embodiment, the YCWP is sterilized.

In specific embodiments, the yeast is suspended in NaOH, including 1M NaOH. In specific embodiments, the first suspension is incubated at about 80° C. for about 1 hour or for 1 hour. In specific embodiments, the centrifuging is performed at about 2000 times gravity for about 10 minutes, or at 2000 times gravity for 10 minutes. In specific embodiments, the pellet is suspended in water, including water at about pH 4.5 or at pH 4.5. In specific embodiments, the second suspension is incubated at about 55° C. for about 1 hour or at 55° C. for 1 hour. In specific embodiments, the pellet is washed in water at least 1, 2, 3 or 4 times. In specific embodiments, the pellet is washed once.

In another embodiment, the YCWP is sterilized using isopropanol and/or acetone following washing of the pellet. In specific embodiments, other known alcohols are appropriate. In specific embodiments, the YCWP is allowed to fully dry after sterilization. In another embodiment, the YCWP is resuspended after being allowed to dry. In specific embodiments, the YCWP is resuspended in PBS, such as 1×PBS.

In another embodiment, the YCWP is allowed to dry and then to be frozen before the antigen is loaded into the YCWP and/or before capped with silicate, in order to place the YCWP in storage before use. In specific embodiments, the YCWP is freeze dried and stored at about 4° C. or lower. In specific embodiments, the YCWP is freeze dried and stored at 4° C.

In another embodiment, the loaded yeast cell wall particle is capped with a silicate. Specifically, in some embodiments the loaded YCWPs are capped by contacting the YCWPs with a silicate, such as tetraalkylorthosilicate, in the presence of ammonia, such that the loaded YCWPs are capped with the silicate. In preferred embodiments, the loaded YCWPs are capped with the silicate within about 60 minutes, about 45 minutes, about 30 minutes, about 15 minutes, about 10 minutes, about 5 minutes or about 2 minutes. The reactivity of the tetraalkylorthosilicates is such that under hydrolysis mediated by the ammonia, the tetraalkylorthosilicates react with the primary hydroxyls of the β-glucan structure of the YCWPs. The tetraalkylorthosilicates also self-react with the ends of these cell wall silicates to form “bridges” such as —O—Si(OH)₂—O— or in three dimensions such as —O—Si(—O—Si—O—)(OH)—O— or —Si(—O—Si—O—)₂—O—. These bridges may occur across the pores in the YCWPs such that the retention of the loaded drug or antigen therein is increased. Such a capped, loaded YCWP can be freeze dried.

The inventor of the present application unexpectedly discovered that loaded YCWPs capped with silicate are an effective vaccine delivery system. More specifically, the capped YCWPs retain more loaded material than the uncapped YCWPs. Even more surprisingly, the capped YCWPs not only deliver significantly more released antigen into the cytoplasm of the phagocytic cells but also deliver significantly more loaded particles into the phagocytic cells in comparison to the uncapped YCWPs.

### Antigen Loaded Yeast Cell Wall Particle

In one embodiment, the antigen is loaded into the particle by incubating the antigen and a suspension of particle, for example, the yeast cell wall particles together and allowing the antigen to penetrate into the hollow insides of the particles.

In another embodiment, after the particle or the yeast cell wall particle is incubated or loaded with the antigen, the combination is freeze-dried to create an anhydrous vaccine within the particle. By freeze-drying, the antigen is trapped within the particle and ready to be phagocytosed by a monocyte, such as a dendritic cell. In specific embodiments, the freeze-drying is the only mechanism used to trap the antigen within the particle. In specific embodiments, the entrapment is not caused by a separate component blocking the antigen from exiting the particle, for example, by physical entrapment, hydrophobic binding, any other binding. In specific embodiments, the entrapment is not caused by crosslinking or otherwise attaching the antigen to the particle outside of any attachment that may occur upon freeze-drying. In specific embodiments, the compositions of the present invention do not include any additional component that specifically assists in evading the lysosome. The antigen includes, for example, a specific protein or a fragment thereof, nucleic acid, carbohydrate, tumor lysate, or a combination thereof.

In another embodiment, the antigen is incorporated into the yeast cell wall particle. In specific embodiments, the number of YCWPs is about 1×10⁹ and the volume of antigen is about 50 μL. In specific embodiments, the incubation is for about one hour or less than one hour at about 4° C. In some embodiments, the combination of YCWPs and antigen is freeze dried over a period of less than or about 2 hours.

In another embodiment, the loaded particle is resuspended in a diluent or solution after the freeze-drying. In specific embodiments, the diluent or solution is water. In specific embodiments, the loaded particle is resuspended and/or incubated with additional antigen, for example, vaccine, to penetrate the particle and the combination is then freeze-dried again. In other embodiments, the combination is subjected to multiple freeze-drying and resuspensions. In other embodiments, the antigen loaded particle is sterilized in ethanol after the freeze-drying and before use.

In specific embodiments, the antigen is loaded into the particle by (a) incubating the antigen and a suspension of the particles, allowing the biological particle to penetrate into the hollow insides of the particles and freeze-drying the suspension of loaded particle and (b) optionally resuspending the particles, incubating the resuspended particles and freeze drying the resuspended particles and any vaccine not already in the particle.

In specific embodiments using YCWPs, the number of YCWPs is about 1×10⁹ and the volume of the antigen is about 50 μL. In specific embodiments, the number of YCWPs is 1×10⁹ and the volume of the antigen is 50 μL. In specific embodiments, the incubation in step (a) is for less than one hour at about 4° C. In specific embodiments, the incubation in step (a) is for about one hour at 4° C. In some embodiments, the foregoing suspension is freeze dried in step (a) over a period of less than 2 hours or over a period of about 2 hours. In some embodiments, the YCWPs in step (b) are resuspended in water, including about 50 μL of water or 50 μL of water. In some embodiments, the resuspended YCWPs are incubated in step (b) for less than or about one hour at about 4° C. or for less than or about 2 hours at 4° C.

Prior to administration, the capped, loaded yeast cell wall particle is resuspended in a pharmaceutically acceptable excipient, such as PBS or a saline solution.

### Methods for Making YCWP Loaded Particles

### (1) Preparing the Antigen

Synthetic antigens such as peptides can be easily produced commercially and provided in lyophilized state. These peptide can be reconstituted and co-incubated with the prepared Yeast Cell Wall Particles (YCWPs) for loading. Similarly, recombinant proteins and/or isolated proteins can be suspended in solution and co-incubated with the YCWPs for loading as discussed below.

### (2) Preparing Yeast Cell Wall Particles

YCWPs were prepared from Fleishmans Baker's Yeast or equivalent. Briefly, 10 g of Fleishmans Baker's yeast was suspended in 100 ml of 1 M NaOH and heated to 80° C. for one hour. The undissolved yeast cell walls were recovered by centrifugation at 2000×g for 10 minutes. The recovered yeast cell walls were then resuspended in 100 ml of water with the pH adjusted to 4.5 with HCl and incubated at 55° C. for an additional hour, and subsequently recovered by centrifugation. The recovered YCWPs were then washed with water once, isopropanol 4 times and finally acetone 2 times. Once the YCWPs were fully dried they were resuspended in PBS, counted, aliquoted into groups of 1×10⁹ particles and freeze dried for use in manufacturing the vaccine.

(3) Loading Antigen into YCWPs

A suspension of fully anhydrous YCWPs (1×10⁹) is placed in contact with 50 μL of a peptide in PBS over a period of 2 hours at 4° C., allowing the peptide to penetrate into the hollow insides of the YCWPs to produce loaded YCWPs. The suspension is then freeze dried for 2 hours. After freeze drying, 50 μL of water is added to the loaded YCWPs, incubated for another 2 hours at 4° C. and again freeze dried to yield YCWPs with dry antigen within their hollow insides. The loaded YCWPs are then sterilized by washing in ethanol and maintained in ethanol.

### (4) Preparing Silicate Capped YCWPs

In a related aspect, the present invention relates to a method for efficient delivery of a vaccine to a subject comprising directly administering to the dermis of the subject a composition comprising (i) a particle and (ii) an antigen selected from a protein, a peptide, an epitope, or an immunogenic fragment, or a subunit thereof, loaded within the particle, as disclosed above. The dermal dendritic cells phagocytose the loaded particle, thereby triggering the immune response to the vaccine.

In specific embodiments, the foregoing method further comprises (a) resuspending the antigen loaded particle in solution and (b) freeze-drying the resuspended solution before step (iii). The antigen comprises a protein, a peptide, an epitope, or an immunogenic fragment, or a subunit thereof.

In specific embodiments, step (iii) comprises: (a) adding an antigen into a yeast cell wall particle, (b) incubating the yeast cell wall particle, (c) freeze-drying the yeast cell wall particle and (d) washing the yeast cell wall, wherein the an antigen comprises a protein, a peptide, an epitope, or an immunogenic fragment, or a subunit thereof, and wherein steps (b)-(c) are repeated at least once with a step of adding water into the yeast cell wall particle before step (b) is repeated.

Yeast cell wall particles (YCWPs) were prepared and loaded with a peptide as described in the examples above. 1 mg of YCWPs were loaded with 500 μg of the peptide. Subsequently, the freeze dried, loaded YCWPs were suspended in 1 ml of absolute ethanol, to which suspension 100 μl of tetraethylorthosilicate and 100 μl of a 10% aqueous ammonia solution were added. The mixture was shaken gently for 15 minutes at room temperature. The YCWPs were then washed thoroughly with absolute ethanol and kept in ethanol at 4° C. until use.

### (5) Administering Loaded YCWPs to Subject

The loaded YCWPs prepared according to Examples above are resuspended in 1 mL of a solution suitable for injection, such as sterile water for injection or sterile saline for injection, which optionally contains 5% human serum albumin, under sterile conditions. Once the loaded YCWPs are carefully resuspended, the entire volume is drawn and injected to the dermis of a patient using a syringe.

### Other Components of the Vaccine Composition

### Adjuvants

The present invention may also comprise one or more adjuvant to boost immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); MPL™ immunostimulant or 3d-MPL (Corixa Corporation); LEIF; salts of calcium, iron or zinc; an insoluble suspension of acylated tyrosine; acylated sugars; cationically or anionically derivatized polysaccharides; polyphosphazenes; aminoalkyl glucosaminide phosphate (ACP); isotucaresol; monophosphoryl lipid A and quil A; muramyl tripeptide phosphatidyl ethanolamine or an imunostimulating complex, including cytokines (e.g., GM-CSF or interleukin-2, -7 or -12) and immunostimulatory DNA sequences. In some embodiments, the adjuvant is selected from the group consisting of monophosphoryl lipid A, CpG oligonucleotides, Poly I:C, Poly ICLC, potent MHC II epitope peptides, and dendritic cell stimulating cytokines such as IL-12, IL-2, and GM-CSF.

### Pharmaceutically Acceptable Salt

The present invention may also comprise one or more pharmaceutically acceptable salt. “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthlalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

### Pharmaceutically Acceptable Carrier

The present invention may also comprise one or more pharmaceutically acceptable carrier. A “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.90%) saline. Compositions comprising such carriers are formulated by well-known conventional methods (see, for example, Remington's Pharmaceutical Sciences, Chapter 43, 14th Ed., Mack Publishing Co, Easton Pa. 18042, USA).

### Method of Treatment

The present invention contemplates both prophylactic and therapeutic uses of the compositions disclosed herein for infectious diseases such as virally-mediated, bacterially-mediated, and parasitic diseases currently targeted with vaccine strategies or those marginally susceptible due to limitations of current vaccine technology. The present invention upon administering to a patient can elicit an effective immune response to the specific antigens and/or to alleviate, reduce, cure or at least partially arrest symptoms and/or complications from the disease or infection. The disease to be treated is not particularly limiting, but depends on the antigen loaded into the particle.

The compositions of the present invention attract phagocytic cells, such as cells of the mononuclear phagocyte system, including monocytes, macrophages, dendritic cells or immature dendritic cells and therefore can be used as a vaccine. In the field of vaccination, cells of the mononuclear phagocyte system are considered “professional” antigen presenting cells and thus, are the ideal target for vaccine delivery. It is well known that presentation of an antigen within an APC is vastly more effective in generating a strong cellular immune response than expression of this same antigen within any other cell type. Therefore, the ability of the compositions of the present invention to present an antigen on an antigen presenting cell via class I MHC and class II MHC molecules dramatically enhances the efficacy of such a vaccine.

The compositions of the present invention come into contact with phagocytic cells either in vivo or in vitro. Hence, both in vivo and in vitro methods are contemplated. As for in vivo methods, the compositions of the present invention are generally administered parenterally, usually intravenously, intramuscularly, subcutaneously, interdermally or intradermally. They may be administered, e.g., by bolus injection or continuous infusion. In in vitro methods, monocytic cells are contacted outside the body and the contacted cells are then parenterally administered to the patient.

### Formulation

The compositions of the present invention may be formulated for mucosal administration (e.g., intranasal and inhalational administration) or for percutaneous administration. The composition of the invention can also be formulated for parenteral administration (e.g., intramuscular, intravenous, or subcutaneous injection), and injected directly into the patient and target cells of monocytic origin, like macrophages and dendritic cells. In specific embodiments, the capped, loaded particles without prior incubation with dendritic cells are directly injected into the dermis of a subject. Thus, the compositions of the present invention may be administered just like a conventional vaccine. This also substantially reduces cost because of the lower level of skill required. In other embodiments, the capped, loaded particle is first incubated with cells of monocytic origin, such as dendritic cells, prior to administration to a subject.

Formulations for injection may be presented in unit dosage form, e.g., in ampules or in multi-dose containers, optionally with an added preservative. The compositions may take such forms as suspensions, solutions or emulsions in oily or aqueous vehicles, and may contain formulatory agents such as suspending, stabilizing and/or dispersing agents. The composition of the present invention may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing, but a suppressive compound may also be used to achieve a tolerogenic response.

### Dosage

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

In some embodiments, an effective or single dose may comprise, e.g., about 10³ to about 10¹³, 10⁴ to about 10⁸, 10⁵ to about 5×10⁷ or about 10⁸ to about 10¹² yeast cell wall particles per kg body weight of the subject. A dose may comprise about 1 to 500 μg, about 500-1,000 μg, about 1 mg-500 mg, about 500 mg to 1,000 mg, or about 1 to 10 g of antigen. Multiple dosages may be used as needed to provide the desired level of protection or treatment. For example, one or more boosters may be needed over time to maintain protection of a eukaryote. Boosters may be given, e.g., every 5-20, 5-10 days, every week, every two weeks, every three weeks, every month or every few months. Boosters may be administered a few times, e.g., 2, 3, 4, 5, 1, 9, 10 or more times. Boosters may also be given one or more months or years after the first administration.

In some embodiments, about 200 μL of a 10×10⁶ concentration of dendritic cells containing locaded particles, or capped, loaded yeast cell wall particles forms one dose of the treatment. In another embodiment, the dose is administered by diluting the 200 μL aliquot to a final volume of 1 ml before administering the dose to a subject. In specific embodiments, the aliquot is diluted with sterile saline containing 5% human serum albumin. In specific embodiments, the 200 μL aliquot will need to be thawed before dilution. In such a scenario, the length of time between thawing and administration of the dose to a subject will be no longer than 2 hours. In some embodiments, the diluted aliquot is administered in a 3 cc syringe. In some embodiments, a syringe needle no smaller than 23 gauge is used.

Regarding the amount of adjuvants, in one embodiment, the amount of one or more immune response enhancing adjuvants is at least about 10 ng, at least about 50 ng, at least about 100 ng, at least about 200 ng, at least about 300 ng, at least about 400 ng, at least about 500 ng, at least about 600 ng, at least about 700 ng, at least about 800 ng, at least about 900 ng, at least about 1 μg, at least about 5 μg, at least about 10 μg, at least about 15 μg, at least about 20 μg, at least about 25 μg, at least about 30 μg, at least about 35 μg, at least about 40 g, at least about 45 μg, at least about 50 μg, at least about 60 μg, at least about 70 μg, at least about 80 μg, at least about 80 μg, at least about 90 μg, or at least about 100 μg. In one embodiment, the amount of adjuvant represents between 1-10% of the composition. The amount of adjuvant is sufficient to stimulate receptors, such as the toll-like receptor, on the dendritic cell.

### Administration

The vaccine of the present invention is typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue. Administration by many of the routes of administration described herein or otherwise known in the art may be accomplished simply by direct administration using a needle, catheter or related device, at a single time point or at multiple time points. In some embodiments, a subject is administered at least 1, 2, 3 or 4 doses of the compositions of the present invention. In specific embodiments, a subject is re-vaccinated once every 4 weeks. In some embodiments, the composition comprising loaded particle is administered to a subject without first fusing to dendritic cells. In specific embodiments, a subject is re-administered with the composition once every 4 weeks. In specific embodiments, about 1-2 million dendritic cells containing the loaded particles or the capped, loaded particles is administered optionally by injection at each vaccination. In specific embodiments, loaded particles or capped, loaded particles are injected in a subject at or near (1) a site of infection or disease, or (2) a lymph node.

### Assaying Vaccination Efficacy

The efficacy of vaccination with the vaccines disclosed herein may be determined in a number of ways.

Vaccine efficacy may be assayed in various model systems. Suitable model systems include a guinea pig model and a mouse model, as described in the examples below. Briefly, the animals are vaccinated and then challenged with a virus or a bacterium. Vaccine may also be administered to already-infected animals. The response of the animals is then compared with control animals. A similar assay could be used for clinical testing of humans. The treatment and prophylactic effects described in the application represent additional ways of determining efficacy of a vaccine.

Vaccine efficacy may further be determined in vitro by viral neutralization assays. Briefly, animals are immunized and serum is collected on various days post-immunization. Serial dilutions of serum are pre-incubated with virus during which time antibodies in the serum that are specific for the virus will bind to it. The virus/serum mixture is then added to permissive cells to determine infectivity by a plaque assay. If antibodies in the serum neutralize the virus, there are fewer plaques compared to the control group.

### EXAMPLES

The present invention is further illustrated by the following examples, which should not be construed as limiting in any way.

### Example 1: Immunization Mice with Yeast Cell Wall Particle Loaded with Recombinant Proteins from N. meningitidis

Two variants of the lapidated protein LP2086 from N. Meningitidis serosubtypes A and B, A05 and B01, were used as bacterial antigens loaded into the YCWPs. Specifically, equal amount of recombinant protein A05 and B01 (10 μg each) were mixed together, then loaded into the yeast cell wall particles and injected subcutaneously into C57 mice. A dosage equivalent to 10 μg A05 protein+10 μg B01 protein was injected in each mouse for immunization. 14 days later, a second injection of the same dose was performed on each mouse. 14 days after the second injection, serum was collected from the tail of each mouse. For regular immunization control, 10 μg A05 protein and 10 μg B01 protein and equal volume of a commercially available alum adjuvant (Imject Alum, Thermo scientific) was injected into another group of C57 mice with the same schedule as the ones immunized with loaded yeast cell wall particles. Serum from mice without vaccination was used as control.

An enzyme-like immunosorbent assay (ELISA) was performed to determine antibody titers against the A05 protein and B01 protein. Specifically, a 96-well Costar plate was coated with 100 μl of recombinant proteins A05 and B01, at a concentration of 10 μg/ml each, by incubation at 4° C. overnight. After washing and blocking, 100 μl of serum from mouse at different dilutions was added to each well. An alkaline phosphatase conjugated secondary antibody and TMB substrate were used for color development. OD at 450 nm was recorded with an ELISA reader.

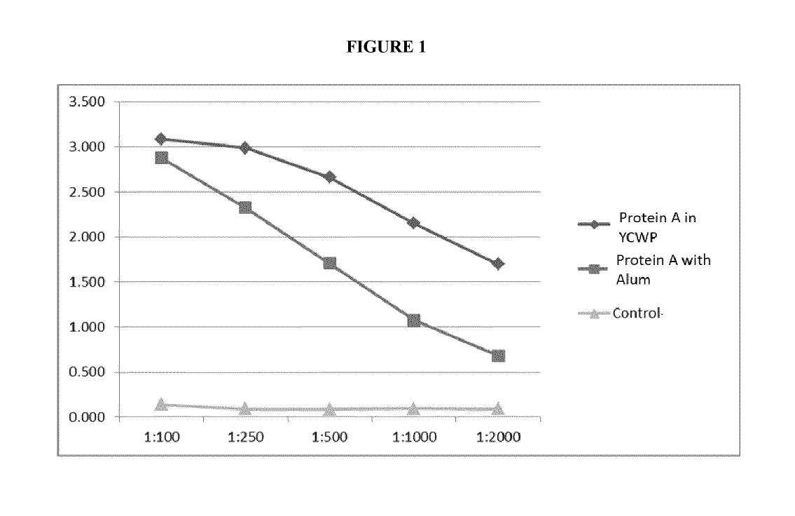

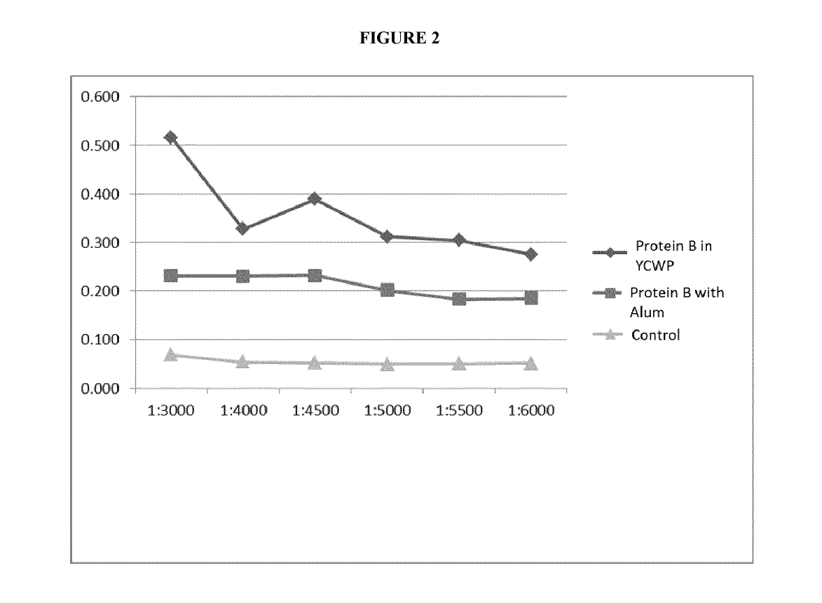

The results shown in FIGS. 1 and 2 indicated that YCWPs loaded with recombinant proteins A05 and B01 induced stronger antibody responses than recombinant proteins with adjuvants. In particular, protein A05 induces antibody response with titers higher than 1:2000 dilution. Protein B01 induces an even stronger antibody response with titles higher than 1:6000 dilution.

### Example 2: Immunization Mice with Yeast Cell Wall Particle Loaded with Recombinant Protein Hemagglutinin from Influenza Virus

The hemagglutinin protein Influenza A virus subtype H5N1 (A/Hong Kong/483/97) was used as viral antigens loaded into the YCWPs. Specifically, the loaded yeast cell wall particles are injected subcutaneously into C57 mice. A dosage equivalent to 10 μg protein was injected in each mouse for immunization. 14 days later, a second injection of the same dose was performed on each mouse. 14 days after the second injection, serum was collected from the tail of each mouse. For regular immunization control, 10 μg protein and equal volume of a commercially available alum adjuvant (Imject Alum, Thermo scientific) was injected into another group of C57 mice with the same schedule as the ones immunized with loaded yeast cell wall particles. Serum from mice without vaccination was used as control.

An enzyme-like immunosorbent assay (ELISA) was performed to determine antibody titers against the hemagglutinin protein. Specifically, a 96-well Costar plate was coated with 100 μl of hemagglutinin protein, at a concentration of 10 μg/ml, by incubation at 4° C. overnight. After washing and blocking, 100 μl of serum from mouse at different dilutions was added to each well. An alkaline phosphatase conjugated secondary antibody and TMB substrate were used for color development. OD at 450 nm was recorded with an ELISA reader.

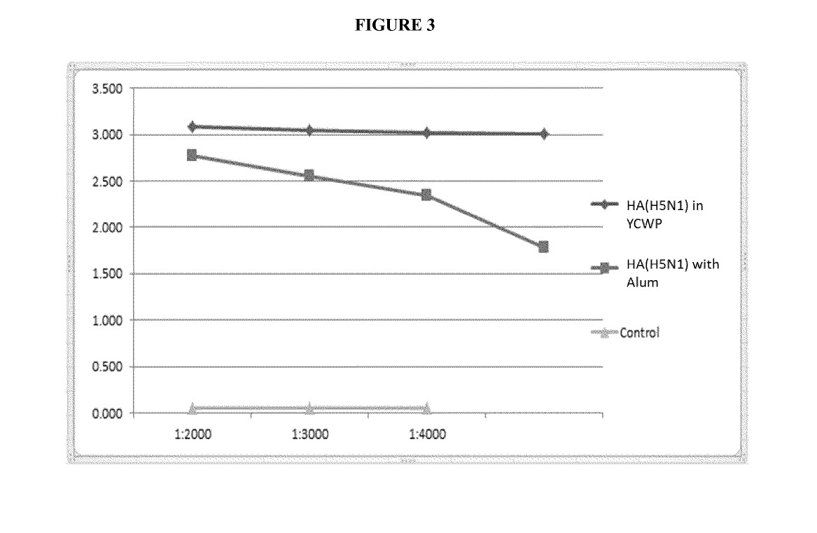

The results shown in FIG. 3 indicated that YCWPs loaded with hemagglutinin protein induced stronger antibody responses than hemagglutinin with adjuvants. In particular, YCWP loaded with hemagglutinin induces antibody response with titers higher than 1:4000 dilution.

### Example 3: T Cell Response with Yeast Cell Wall Particle Loaded with Recombinant Proteins from N. meningitidis

The T cell response from YCWPs loaded with N. meningitidis proteins A05 and B01 is monitored with mixed lymphocyte reaction (MLR). Specifically, peritoneal macrophages or bone marrow dendritic cells are isolated from C57 mice (the same strain of mice for immunization) and cultured in 96-well plates. YCWPs loaded with recombinant proteins B01 and A05 are then added to the cell culture at a ratio of about 10 loaded YCWPs per macrophage or dendritic cell. After an overnight incubation, 2×10⁵ lymphocytes or splenocytes from mice immunized with protein A05 and B01 loaded YCWPs is added to the culture and the co-culture will be maintained for another 72 hours. At the end of culture, MTT dye solution from CellTiter 96 Non-Radioactive Cell Proliferation Assay kit (Promega) is added to each well. The plate is incubated at 37° C. for up to 4 hours in a humidified, 5% CO₂ atmosphere and the absorbance at 570 nm wavelength is recorded. As controls, medium alone, macrophage/dendritic cell alone, and lymphocyte alone are used. The average of the absorbance values in medium alone wells (negative control) is used as a blank value and subtracted from all absorbance values to yield corrected Absorbance Values.

In this assay, YCWPs loaded macrophages or dendritic cells function as antigen presenting cells. When lymphocytes or splenocytes from mice immunized with the same beads come in contact with these antigen presenting cells, the cytotoxic T lymphocytes against the target proteins within the lymphocytes or splenocytes will be stimulated by these antigen presenting cells to proliferate. If the immunization is not successful, there will be no cell proliferation due to lack of specific cytotoxic T lymphocytes. It is expected that the YCWPs loaded with recombinant proteins A05 and B01 will stimulate cell proliferation, which will produce a strong UV absorption. In contrast, the controls (medium alone, macrophage/dendritic cell alone, and lymphocyte alone) will produce minimum UV absorption, indicating no cell proliferation.

### Example 4: T Cell Response Assay for YCWPs Loaded with Hemagglutinin Protein of Influenza A

To produce influenza vaccines, YCWPs are loaded with hemagglutinin (HA) recombinant protein from Influenza A Virus subtype H5N1 (A/Hong Kong/483/97). T cell response from Influenza A virus vaccine is monitored by mixed lymphocyte reaction (MLR). Specifically, peritoneal macrophages or bone marrow dendritic cells are isolated from C57 mice (the same strain of mice for immunization) and cultured in vitro in 96-well plates. The loaded YCWPs are then be added to the culture at a ratio of about 10 YCWPs/macrophage or dendritic cell. After overnight culture, 2×10⁵ lymphocytes or splenocytes from immunized mice are added to the culture and the co-culture will be maintained for another 72 hours. At the end of culture, MTT dye solution from CellTiter 96 Non-Radioactive Cell Proliferation Assay kit (Promega) is added to each well. The cell culture is incubated at 37° C. for up to 4 hours in a humidified, 5% CO₂ atmosphere and the absorbance at 570 nm wavelength is recorded. As controls, medium alone, macrophage/dendritic cell alone, and lymphocyte alone are used. The average of the absorbance values in medium alone wells (negative control) is used as a blank value and subtracted from all absorbance values to yield corrected Absorbance Values.

It is expected that the YCWPs loaded with recombinant proteins hemagglutinin from Influenza A virus will produce a strong UV absorption, indicating a strong cell proliferation. In contrast, the controls (medium alone, macrophage/dendritic cell alone, and lymphocyte alone) will produce minimum UV absorption, indicating no cell proliferation.

### Example 5: T Cell Response Assay for YCWPs Loaded with HIV gp120 Protein

To produce HIV vaccines, YCWPs are loaded with Envelope glycoprotein gp120 from HIV. The T cell response from gp120 loaded YCWPs is measured with mixed lymphocyte reaction (MLR) and enzyme-linked immunospot (ELISPOT) assays. Specifically, MLR is used to determine CD4 cell response, and ELISPOT assay is used to determine CD8 response. Briefly, 96-well nitrocellulose plates (Millipore Corp., Bedford, Mass.) are coated with 100 ul of phosphate-buffered saline (PBS) containing 5 ug/ml of anti-mouse gamma interferon (IFN-Y) monoclonal antibody. After incubation at 4° C. overnight, the wells are washed eight times with DMEM-high glucose medium containing 10% FBS and incubated for more than 1 h at 37° C. Two-fold dilution series of splenocytes, starting at 5×10⁵ cells per well, are placed into the coated wells and co-cultured with peritoneal cavity macrophages or bone marrow dendritic cells which have been fed with gp120 loaded YCWPs. Unloaded macrophage or dendritic cells are used as negative controls. The plates are incubated in a 5% CO₂ incubator for 30 h. Subsequently, the plates are extensively washed with PBS-Tween 20 (0.05%) and then 0.1 ml of 2.5 mg/ml of biotinylated anti-mouse IFN-Y monoclonal antibody is added to each well. After incubation overnight at 4° C., the plates are incubated with peroxidase-labeled streptavidin for 1 h at room temperature. Wells are washed with PBS Tween-20 and PBS, and substrate (3,3′-diaminobenzidine tetrahydrochloride) at a concentration of 1 mg/ml and containing 0.015% hydrogen peroxidase in 50 mM Tris-HCl, pH 7.5, is added. The spots are counted.

It is expected that the YCWPs loaded with HIV gp120 will produce a strong UV absorption, indicating a strong cell proliferation and T cell response. In contrast, the controls (unloaded macrophase or dendritic cells) will produce minimum UV absorption, indicating no cell proliferation and no T cell response.

### Example 6: Immunization of Mice with a Yeast Cell Wall Particle Loaded with Recombinant HIV gp120 Protein

HIV envelope glycoprotein gp120 is used as an antigen loaded into the YCWPs. The yeast cell wall particles loaded with gp120 is injected subcutaneously into mice for evaluating immune response. A dosage equivalent to 10 μg gp120 protein is injected in each mouse for immunization. 14 days later, a second injection of the same dose is performed on each mouse. 14 days after the second injection, serum is collected from the tail of each mouse. For regular immunization control, 10 μg gp120 protein and equal volume of a commercially available alum adjuvant (e.g. Imject Alum, Thermo scientific) is injected into another group of mice with the same schedule as the ones immunized with loaded yeast cell wall particles. Serum from mice without vaccination is used as control.

An enzyme-like immunosorbent assay (ELISA) is performed to determine antibody titers against the gp120 protein. Specifically, a 96-well Costar plate is coated with 100 μl of recombinant proteins gp120, at a concentration of 10 μg/ml, by incubation at 4° C. overnight. After washing and blocking, 100 μl of serum from mouse at different dilutions is added to each well. An alkaline phosphatase conjugated secondary antibody and TMB substrate are used for color development. OD at 450 nm is recorded with an ELISA reader.

It is expected that YCWPs loaded with HIV gp120 will induce stronger antibody responses than recombinant proteins with adjuvants, which will be reflected by a higher titer.

### Example 7: T Cell Response Assays for YCWPs Loaded with Recombinant HIV gp120 Protein

Non-Radioactive LDH Cytotoxicity Assays

A target cell line expressing HIV gp120 is established first in B16 melanoma cells. Specifically, synthetic gp120 gene sequence is cloned into pcDNA3.1 to obtain a DNA construct pcDNA3.1/HIV gp120, which is then used to transfect B16 melanoma cells with lipofectamine 2000. After G418 selection, clones are screened with RT-PCR and Western blot analysis. High gp120 expression clones are selected as target cell lines (B16/gp120) for Non-radioactive LDH cytotoxicity assay.

Next, splenocytes from mice immunized with an HIV vaccine are isolated with standard procedures. Splenocytes are seeded into 24-well plates at a concentration of 4×10⁶ cells/well in RPMI 1640 medium with Glutamax-I (Gibco) supplemented with 100 U/mL penicillin, 100 mg/mL streptomycin, and 10% heat-inactivated FBS, plus 10 U/mL of human interleukin 2. Then, gp120 protein is added to the culture to a final concentration of 5×10⁻⁷ M. The culture is maintained in a humidified 5% CO₂ incubator at 37° C. for 7 days. On the 6^{th} day, B16/gp120 target cells are plated into 96-well plates at a concentration of 1.5×10⁴ per well and cultured overnight. On the 7^{th} day, splenocytes in 24 well plates are harvested and washed. The splenocytes are re-suspended and added to the B16/gp120 target cell culture at different effector/target (E:T) ratios. The procedures generally follow the protocol recommended by the CytoTox 96® Non-Radioactive Cytotoxicity Assay kit (Promega). The CytoTox 96® assay quantitatively measures the concentration of lactate dehydrogenase (LDH), a stable cytosolic enzyme that is released upon target cell lysis. As controls, medium alone, effector and target cells alone (spontaneous LDH release), and target cell completely lysed by detergent (maximum LDH release). The antigen specific toxicity of CD8 T cells is represented by the percentage cytotoxicity, which is calculated as follows:

$$\% Cytotoxicity = \frac{Experimental - Effector Spontaneous - Target Spontaneous}{Target Maximum - Target Spontaneous} \times 100$$

Flow Cytometry Assay for Detecting Antigen-Specific CD8+ T Cells

This assay measures cell surface CD107a and CD107b, which are normally present in the membrane of cytotoxic granules formed a result of degranulation.

About 1×10⁶ splenocytes isolated from mice immunized with YCWP loaded with gp120 are incubated with 1 pig/ml each of anti-CD28 and anti-CD49d and 2 μg/ml of gp120 protein at a total volume of lml. PE or FITC conjugated antibodies against CD107a and CD107b are added to the cells before stimulation. The cultures are incubated for 1 h at 37° C. in a 5% CO₂ incubator, followed by an additional 4-5 h incubation in the presence of the secretion inhibitor monensin (BDPharmingen). Right after stimulation, splenocytes are washed once, and stained with conjugated antibodies against CD8. The cells are washed and then fix and permeabilized. After permeabilization, the cells are washed twice, and stained directly with conjugated antibodies that are specific for intracellular markers IFN-γ. The cells are then washed for a final time and resuspended in 1% paraformaldehyde in PBS. Cells of CD8⁺/CD107a⁺/CD107b⁺ or CD8⁺/CD107a⁺/CD107b⁺/IFNg⁺ are analyzed by flow cytometry, the procedure of which is for example described by Betts et al., Journal of Immunological Methods (2003), 281:65-78.

This assay measures the percentage of antigen activated CD8 T cells as compared to the non-activated CD8 T cells. The activated CD8 T cells are isolated based on flow cytometry because they have the markers CD8⁺/CD107a⁺/CD107b⁺ or CD8⁺/CD107a⁺/CD107b⁺/IFNg⁺.

In Vitro Lymphocyte Proliferation Assay

For this assay, splenocytes from mice immunized with YCWP loaded with gp120 are isolated with standard procedures. The isolated splenocytes are then stimulated with 1 μg/ml of gp120 for 5 days. The stimulated splenocytes are used with the CellTiter 96® Non-Radioactive Cell Proliferation Assay following the manufacturer's protocol (Promega).

In particular, this assay measures the conversion of ([3-(4,5-dimethylthiazol-2-yl)-5-(3carboxymethoxyphenyl)-2-(4-sulfophenyl)-2H-tetrazolium, inner salt]) (MTS) into formazan by dehydrogenases from metabolically active cells. The formazan concentration can be measured by absorption at 490 nm, which is proportionate to the number of living cells in culture. The antigen specific T cell proliferation is expressed as percentage increase of absorbance at 490 nm in gp120 stimulated splenocytes over non-stimulated splenocytes.

Intracellular Cytokine Staining

Splenocytes from mice immunized with HIV vaccine are isolated with standard procedures. The isolated splenocytes are stimulated in vitro with gp120 protein in the presence of anti-CD28 and anti-CD49d antibodies. After 2 h of incubation at 37° C., BrefeldinA is added to the culture to inhibit cytokine secretion, and the culture is then incubated overnight. Cells are subsequently harvested, stained for surface CD4+ and then fixed. The fixed cells are then permeabilized and stained with labeled antibodies against IL-2 and IFN-γ. CD4⁺/IL2⁺ and or IFNg⁺ cells are analyzed with flow cytometry.

This assay measures the percentage of antigen activated CD4 T cells as compared to the non-activated CD4 T cells. The activated CD4 T cells are isolated based on flow cytometry because they have the markers CD4⁺/IL2⁺, CD4⁺/IFNg⁺ or CD4⁺/IL2⁺/IFNg⁺.

1. A vaccine comprising (i) a yeast cell wall particle, and (ii) an antigen loaded into the yeast cell wall particle, wherein the vaccine upon administration to a human stimulates an immune response.

2. A vaccine according to claim 1, wherein the antigen is selected from the group consisting of viral antigens and bacterial antigens.

3. A vaccine according to claim 2, wherein the antigen is bacterial antigen.

4. A vaccine according to claim 3, wherein the antigen is a protein derived from N. Meningitidis, or a fragment thereof.

5. A vaccine according to claim 4, wherein the antigen is recombinant protein A05 or B01 from N. meningitidis, or a combination thereof.

6. A vaccine according to claim 1, wherein the antigen is viral antigen.

7. A vaccine according to claim 6, wherein the antigen is a protein derived from influenza A, or a fragment thereof.

8. A vaccine according to claim 7, wherein the antigen is hemagglutinin of influenza A, or a fragment thereof.

9. A vaccine according to claim 6, wherein the antigen is a protein derived from HIV, or a fragment thereof.

10. A vaccine according to claim 9, wherein the antigen is gp120 of HIV, or a fragment thereof.

11. A vaccine according to claim 1, wherein the yeast cell wall particle further comprises a silicate.

12. A vaccine according to claim 11, wherein the yeast cell wall particle is modified by capping with the silicate.

13. A vaccine according to claim 11, wherein the silicate comprises an organic moiety attached to each of the four oxygen compounds of an orthosilicate.

14. A vaccine according to claim 11, wherein the silicate is selected from the group consisting of tetraethylorthosilicate, tetramethylorthosilicate, tetrapropylorthosilicate, and tetrabutylorthosilicate.

15. A vaccine according to claim 1, further comprising one or more adjuvants, excipients and preservatives.

16. A vaccine according to claim 15, wherein the adjuvants are loaded within the yeast cell wall particle.

17. A vaccine according to claim 16, wherein the adjuvant is monophosphoryl lipid A or CpG oligonucleotide.

18. A method for efficiently delivering a vaccine to a subject comprising administering a vaccine according to claim 1.

19. The method of claim 18, wherein the vaccine is administered subcutaneously, orally, or intravenously.

20. The method of claim 19, wherein the vaccine is directly administered to the dermis of the subject.


SEQUENCE LISTING (4 sequences)

SEQ ID NO: 1 - protein, 268 aa, Artificial Sequence
  Description of Artificial Sequence Synthetic polypeptide
MCSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQHHHHHH

SEQ ID NO: 2 - protein, 267 aa, Artificial Sequence
  Description of Artificial Sequence Synthetic polypeptide
MCSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQHHHHHH

SEQ ID NO: 3 - protein, 555 aa, Influenza A virus
MEKIVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGKSSFFRNVVWLIKKNSTYPTIKRSYNNTNQEDLLVLWGIHHPNDAAEQTKLYQNPTTYISVGTSTLNQRLVPEIATRPKVNGQSGRIEFFWTILKPNDAINFESNGNFIAPEYAYKIVKKGDSTIMKSELEYGNCNTKCQTPMGAINSSMPFHNIHPLTIGECPKYVKSNRLVLATGLRNAPQRERRRKKRGLFGAIAGFIEGGWQGMVDGWYGYHHSNEQGSGYAADQESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSLYSTVASSLALAIMVAGLSLW

SEQ ID NO: 4 - protein, 6 aa, Artificial Sequence
  Description of Artificial Sequence Synthetic 6xHis tag
HHHHHH